(19) United States
(12) Patent Application Publication
(10) Pub. No.: US 2023/0260593 A1
(43) Pub. Date: Aug. 17, 2023

(54) DEEP LEARNING-BASED ANTIBIOTIC RESISTANCE GENE PREDICTION SYSTEM AND METHOD

(71) Applicant: KING ABDULLAH UNIVERSITY OF SCIENCE AND TECHNOLOGY, Thuwal (SA)

(72) Inventors: Xin GAO, Thuwal (SA); Yu LI, Thuwal (SA); Wenkai HAN, Thuwal (SA)

(21) Appl. No.: 17/768,332

(22) PCT Filed: May 22, 2020
(86) PCT No.: PCT/IB2020/054897

§ 371 Date: Apr. 12, 2022

Related U.S. Application Data
(60) Provisional application No. 62/916,345, filed on Oct. 17, 2019.
(60) Provisional application No. 62/915,162, filed on Oct. 15, 2019.

(51) Int. Cl. G16B 20/00; G16B 40/20; G06N 3/08

(52) U.S. Cl. CPC G16B 20/00; G16B 40/20; G06N 3/08

(57) ABSTRACT

A method for annotating antibiotic resistance genes includes receiving a raw sequence encoding of a bacterium, determining first, in a level 0 module, whether the raw sequence encoding includes an antibiotic resistance gene (ARG), determining second, in a level 1 module, a resistant drug type, a resistance mechanism, and a gene mobility for the ARG, determining third, in a level 2 module, if the ARG is a beta-lactam, a sub-type of the beta-lactam, and outputting the ARG, the resistant drug type, the resistance mechanism, the gene mobility, and the sub-type of the beta-lactam. The level 0 module, the level 1 module and the level 2 module each includes a deep convolutional neural network (CNN) model.

### CROSS-REFERENCE TO RELATED APPLICATIONS

This application claims priority to U.S. Provisional Patent Application No. 62/915,162, filed on Oct. 15, 2019, entitled “A DEEP LEARNING-BASED ANTIBIOTIC RESISTANCE GENE PREDICTION FRAMEWORK,” and U.S. Provisional Patent Application No. 62/916,345, filed on Oct. 17, 2019, entitled “A DEEP LEARNING-BASED ANTIBIOTIC RESISTANCE GENE PREDICTION FRAMEWORK,” the disclosures of which are incorporated herein by reference in their entirety.

### BACKGROUND

### Technical Field

Embodiments of the subject matter disclosed herein generally relate to an end-to-end, hierarchical, multi-task, deep learning system for antibiotic resistance gene (ARG) annotation, and more particularly, to a system that is capable of ARG annotation by taking raw sequence encoding as input and then annotating ARGs sequences based on three aspects: resistant drug type, the underlying mechanism of resistance, and gene mobility.

### Discussion of the Background

The abuse of antibiotics in the last several decades has given rise to wide spread antibiotic resistance. This means that infecting bacteria are able to survive the exposure to antibiotics which can normally kill them. There are indications that this problem has become one of the most urgent threats to the global health. To investigate its properties and thus combat it, at the gene level, researchers are trying to identify and study antibiotic resistance genes (ARGs). However, to handle the computational challenges posed by the enormous amount of data in this field, some tools, such as DeepARG (Arango-Argoty et al., 2018), AMRFinder (Feldgarden et al., 2019), ARGs-OAP (sARG) (Yin et al., 2018) and ARG-ANNOT (Gupta et al., 2014), have been developed to help people identify and annotate ARGs. Despite the wide usage of these tools, however, almost all the existing tools, including DeepARG, which utilizes sequence alignment to generate features, rely heavily on the sequence alignment and comparison against the existing ARGs databases.

New sequencing technologies have greatly reduced the cost for sequencing bacterial genomes and metagenomes and have increased the likelihood of rapid whole-bacterial-genome sequencing. The number of genome releases has increased dramatically and many of these genomes have been released into the public domain without publication, and their annotation relies on automatic annotation mechanisms. Rapid Annotation using Subsystem Technology (RAST) is one of the most widely used servers for bacterial genome annotation. It predicts the open reading frames (ORFs) followed by annotations. Although RAST is widely used, it annotates many novel proteins as hypothetical proteins or restricts the information to the domain function. RAST also provides little information about antibiotic resistance genes (ARG). Information on resistance genes can be found in the virulence section of an annotated genome or can be extracted manually from the generated Excel file using specific key words. This process is time-consuming and exhaustive. The largest barrier to the routine implementation of whole-genome sequencing is the lack of automated, user-friendly interpretation tools that translate the sequence data and rapidly provide clinically meaningful information that can be used by microbiologists. Moreover, because released sequences are not always complete sequences (for both bacterial genomes and metagenomes), sequence analysis and annotation should be performed on contigs or short sequences to detect putative functions, especially for ARGs.

Several ARG databases already exist, including Antibiotic Resistance Genes Online (ARGO), the microbial database of protein toxins, virulence factors, and antibiotic resistance genes MvirDB, Antibiotic Resistance Genes Database (ARDB), Resfinder, and the Comprehensive Antibiotic Resistance Database (CARD). However, these databases are neither exhaustive nor regularly updated, with the exception of ResFinder and CARD. Although ResFinder and CARD are the most recently created databases, the tools associated with these databases are located in a website, focus only on acquired AR genes, and do not allow the detection of point mutations in chromosomic target genes known to be associated with AR.

In addition to the two disadvantages mentioned in Arango-Argoty et al. (2018) with regard to the existing tools, that is, the sequence alignment can cause high false negative rate and be biased to specific types of ARGs due to the incompleteness of the ARGs databases, those tools also require careful selection of the sequence alignment cutting-off threshold, which can be difficult for the users who are not very familiar with the underlying algorithm.

Moreover, except CARD, most of those tools are uni-functional, i.e., they can only annotate the ARGs from a single aspect. They can either annotate the resistant drug type or predict the functional mechanism. Together with the gene mobility property, which describes whether the ARG is intrinsic or acquired, all of those different pieces of information are useful to the users. Thus, it is desirable for the community to first construct a database, which contains multi-task labels for each ARG sequence, and then develop a method, which can perform the above three annotation tasks simultaneously.

Thus, there is a need for a new system, server and method that is capable to annotate a given ARG from three different aspects: resistant drug type, mechanism, and gene mobility.

### BRIEF SUMMARY OF THE INVENTION

According to an embodiment, there is a method for annotating antibiotic resistance genes and the method includes receiving a raw sequence encoding of a bacterium; determining first, in a level 0 module, whether the raw sequence encoding includes an antibiotic resistance gene (ARG); determining second, in a level 1 module, a resistant drug type, a resistance mechanism, and a gene mobility for the ARG; determining third, in a level 2 module, if the ARG is a beta-lactam, a sub-type of the beta-lactam, and outputting the ARG, the resistant drug type, the resistance mechanism, the gene mobility, and the sub-type of the beta-lactam. The level 0 module, the level 1 module and the level 2 module each includes a deep convolutional neural network (CNN) model.

According to another embodiment, there is a server for annotating antibiotic resistance genes. The server includes an interface for receiving a raw sequence encoding of a bacterium, and a processor connected to the interface. The processor is configured to determine first, in a level 0 module, whether the raw sequence encoding includes an antibiotic resistance gene (ARG); determine second, in a level 1 module, a resistant drug type, a mechanism, and a gene mobility for the ARG; determine third, in a level 2 module, if the ARG is a beta-lactam, a sub-type of the beta-lactam, and output the ARG, the resistant drug type, the mechanism, the gene mobility, and the sub-type of the beta-lactam. The level 0 module, the level 1 module and the level 2 module each includes a deep convolutional neural network (CNN) model.

According to still another embodiment, there is a hierarchical, multi-task, deep learning model for annotating antibiotic resistance genes, and the model includes an input for receiving a raw sequence encoding of a bacterium; a level 0 module configured to determine first, whether the raw sequence encoding includes an antibiotic resistance gene (ARG); a level 1 module configured to determine second, a resistant drug type, a mechanism, and a gene mobility for the ARG; a level 2 module configured to determine third, if the ARG is a beta-lactam, a sub-type of the beta-lactam, and an output configured to output (708) the ARG, the resistant drug type, the mechanism, the gene mobility, and the sub-type of the beta-lactam. The level 0 module, the level 1 module and the level 2 module each includes a deep convolutional neural network (CNN) model.

### BRIEF DESCRIPTION OF THE DRAWINGS

For a more complete understanding of the present invention, reference is now made to the following descriptions taken in conjunction with the accompanying drawings, in which:

FIG. 1 is a schematic diagram of a Hierarchical Multi-task Deep learning server for annotating Antibiotic Resistance Genes;

FIG. 2 is a schematic diagram of a Deep Convolutional Neural Network used in the Hierarchical Multi-task Deep learning server;

FIG. 3 is a schematic diagram of a convolutional layer used in the Deep Convolutional Neural Network;

FIG. 4 is a schematic diagram of a level 1 module used in the Hierarchical Multi-task Deep learning server for determining a drug target, resistance mechanism, and mobility of a sequence;

FIG. 5 illustrates the structure of a database that includes three kinds of annotations: drug target, mechanism of antibiotic resistance, and transferable ability;

FIG. 6 illustrates possible values for the hyperparameters of the Deep Convolutional Neural Networks used in the Hierarchical Multi-task Deep learning server;

FIG. 7 is a flowchart of a method for annotating Antibiotic Resistance Genes;

FIG. 8 is a table that presents results when various existing tools are compared to the results produced by the Hierarchical Multi-task Deep learning server;

FIG. 9 illustrates the number of predicted ARG by the Hierarchical Multi-task Deep learning server versus the existing tools;

FIG. 10 illustrates the results obtained with the Hierarchical Multi-task Deep learning server when applied to validation data that comes from different North American soil samples; and

FIG. 11 is a schematic illustration of the configuration of a computing system in which the Hierarchical Multi-task Deep learning server can be implemented.

### DETAILED DESCRIPTION OF THE INVENTION

The following description of the embodiments refers to the accompanying drawings. The same reference numbers in different drawings identify the same or similar elements. The following detailed description does not limit the invention. Instead, the scope of the invention is defined by the appended claims. The following embodiments are discussed, for simplicity, with regard to a server, which based on deep learning, is capable to annotate a given ARG from three different aspects: resistant drug type, mechanism and gene mobility. With the help of hierarchical classification and multi-task learning, the server can achieve the state-of-the-art performance on all the three tasks. However, the embodiments to be discussed next are not limited to deep learning, but may be implemented with other solvers.

Reference throughout the specification to “one embodiment” or “an embodiment” means that a particular feature, structure or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments.

According to an embodiment, a novel server, called herein Hierarchical Multi-task Deep learning for annotating Antibiotic Resistance Genes (HMD-ARG), is introduced to solve the above problems and meet the needs of the community. The server is believed to include the first multi-task dataset in this field and to provide the first service to annotate a given ARG sequence from three different aspects with multi-task deep learning. Regarding the dataset, in one embodiment, all the existing ARGs datasets were merged to construct the largest datasets in the market for the above three tasks. Then, the three labels for each sequence were aggregated based on the header and the sequence identity. After the above processing (more details are discussed next), a multi-task dataset for ARG annotation was generated.

According to this embodiment, the algorithm underlying the server relies on hierarchical multi-task deep learning (see, for example, Li et al., 2019, 2017; Zou et al., 2019) without utilizing sequence alignment as the other algorithms do. Unlike DeepARG, the novel HMD-ARG model directly operates upon the ARG raw sequences instead of the similarity scores, which can potentially identify useful information or motifs omitted by the existing sequence alignment algorithms. Further, with just one model instead of three, given an ARG sequence, the novel HMD-ARG model can simultaneously predict its resistant drug type, its functional mechanism, and whether it is intrinsic or acquired ARG. For this task, the labeling space has a hierarchical structure. That is, given a sequence, it can first be classified into ARG or non-ARG. If it is an ARG, the HMD-ARG model can identify its coarse resistant drug type. If the drug is a β-lactam, the HMD-ARG model can further predict its detailed subtypes. Based on the above structure, the HMD-ARG model was designed to use a hierarchical classification strategy to identify ARG, annotate ARG coarse type and predict ARG sub-type, sequentially. With the help of the above three designs, the server that implements the novel HMD-ARG model can not only perform the most comprehensive annotation for ARG sequences, but it also can achieve the state-of-the-art performance on each task with a reasonable running time.

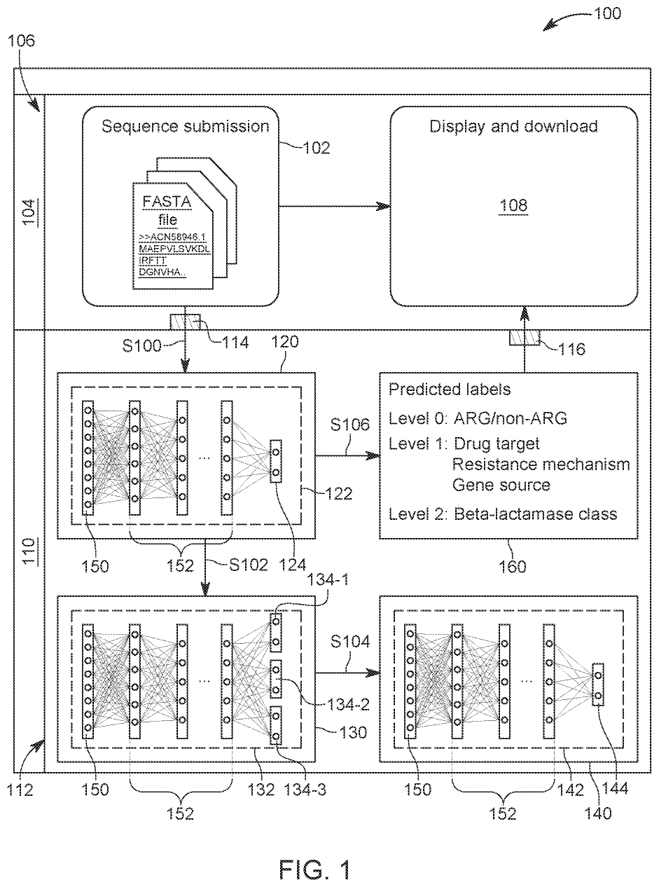

The HMD-ARG model is now discussed in more detail with regard to the figures. The HMD-ARG model 110, which is part of a system 100, as shown in FIG. 1, has three modules 120, 130, and 140, with a hierarchical structure. Each of the three modules uses a corresponding Deep Convolutional Neural Network (CNN) [Krizhevsky, 2012] model, which is indicated with reference numbers 122, 132, and 142, respectively. The CNN models 122, 132, and 142 are identical, except for the output layer. More specifically, as schematically illustrated in FIG. 1, each of the CNN models 122, 132, and 142 have an input layer 150, plural hidden layers 152, and an output layer 124 for the first module 120, three output layers 134-1 to 134-3 for the second module 130, and one output layer 144 for the third module 140.

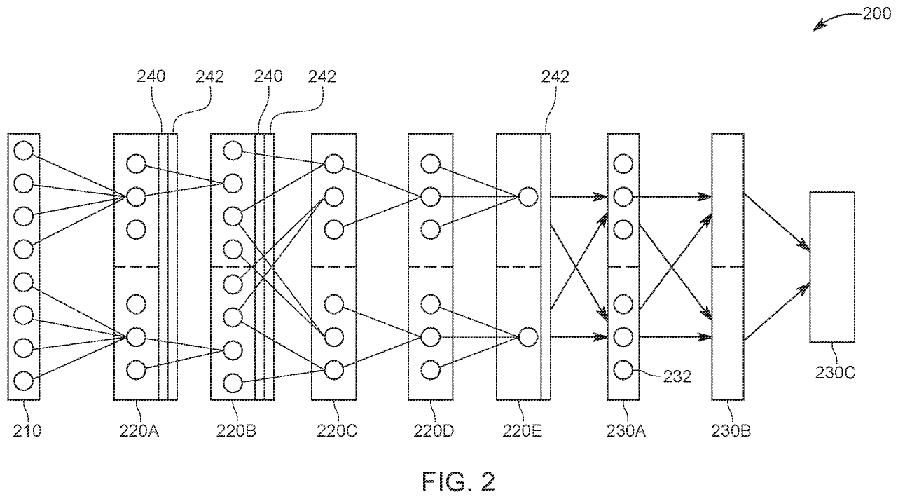

A possible CNN model 200 that is common to the CNN models 122, 132, and 142 may include, as illustrated in FIG. 2, an input layer 210, five convolutional layers 220A to 220E, and three fully-connected layers 230A to 230C. The five convolutional layers and the three fully-connected layers are learned layers and also have weights. FIG. 2 shows that the five convolutional layers may be implemented as two separate blocks, each run on a different machine of the system 100. In one application, the kernels of the second, fourth and fifth convolutional layers 220B, 220D and 220E are connected only to those kernel maps in the previous layer which reside on the same machine (e.g., CPU or GPU), while the kernel of the third convolutional layer 220C is connected to all kernel maps in the second layer 220B, indifferent of the machine. The neurons 232 in the fully-connected layers 230A to 230C are connected to all the neurons in the previous layer. Response-normalization layers 240 may follow the first and second convolutional layers 220A and 220B. Max-pooling layers 242 follow both the response-normalization layers 240 as well as the firth convolutional layer 220E, as schematically shown in FIG. 2.

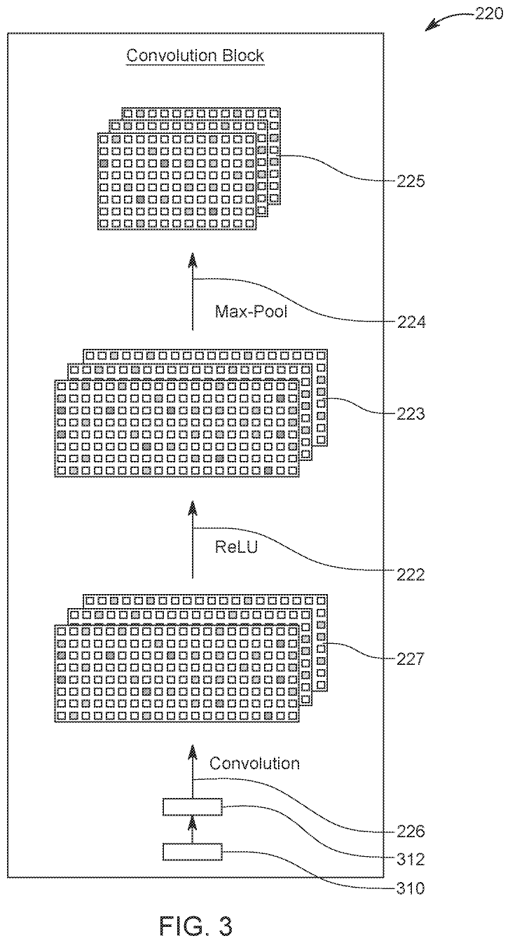

A possible implementation of a convolutional layer 220A to 220E is illustrated in FIG. 3 and may have a number of convolution filters which become automatically-learned feature extractors after training. A rectified linear unit (ReLU) 222 is used as the activation function. The max-pooling operation 224 after that allows only values from highly-activated neurons to pass to the upper fully-connected layers 230A to 230C. The three operations of the convolution block 220 include the convolution operation 226, the ReLU 222, and the max-pooling 224. The CNN 220 in FIG. 3 receives a genomic sequence 310 and applies a one-hot encoding 312 to it. The encoded data is then convolved in a convolution step 226, which results in convolved data 227. The convolved data is modeled with the ReLU units in step 222 to obtain the processed data 223, and the processed data 223 is applied to the Max-Pool layer 224 to summarize the outputs of neighboring group of neurons in the same kernel map and generate the output data 225. The output data 225 is then provided to the fully-connected layers 230A to 230C. Those skilled in the art would understand that the examples shown in FIGS. 2 and 3 can be modified to use more or less layers.

Returning to FIG. 1, it is noted that although the CNN models 122, 132, and 142 are identical, the output layers 124, 134, and 144 are different for the modules 120, 130, and 140. For the level 0 module 120 and the level 2 module 140, the output layer is a single layer while for the level 1 module 130, the output layer includes three task-specific output layers 134-1 to 134-3.

Further, FIG. 1 shows that input data 102 is provided by a user to the HMD-ARG model 110 in step S100, at an input 114. The input data 102 may include a raw sequence encoding of a bacterium, in a given format that is used in the art. The user may input this data from a browser 104 that is residing on a remote computer 106 (belonging to the user) while the HMD-ARG model 110 is implemented on a server 112, which is remote from the user. The received input data 102 is provided to the level 0 module 120, for determining the ARG or non-ARG nature of the input. The input data 102 is provided to the input layer 150 of the CNN model 122 and the output layer 124 provides this indication.

Then, if the result of the determination of the level 0 module 120 was that the provided sequence is ARG, the same input is provided in step S102 to the level 1 module 130, for determining the coarse resistant drug type. If the result of this step is that the ARG is a β-lectern, then, in step S104, an output from the level 1 module 130 is provided to the level 2 module 140, together with the input data 102, for determining the β-lactamase class.

The level 2 module 140 generates the β-lactamase class and this information together with the ARG/non-ARG determination, and the drug target, resistance mechanism, and the gene mobility are provided in step S106 as a predicted table output 160. The output information 160 is provided by the server 112, through the output 116, back to the user's terminal 106, in the browsers 104, as an output file 108 that has all the information of the predicted table output 160.

Taking advantage of the above structure, the hierarchical HMD-ARG model 110 performs the above three predictions in sequential order. This hierarchical framework helps the model HMD-ARG 110 deal with the data imbalance problem (Li et al., 2017 Li, Y., Wang, S., Umarov, R., Xie, B., Fan, M., Li, L., and Gao, X. DEEPre: sequence-based enzyme EC number prediction by deep learning. Bioinformatics, 34(5), 760-769.) and save the computational power for non-ARG. Note that the multi-task learning model shown in FIG. 1 is designed for the coarse resistant drug type prediction, and mechanism as well as gene mobility predictions.

For the deep learning models of the HMD-ARG model 110, the inputs are proteins sequences, which are strings composed of 23 characters representing different amino acids. To make the inputs suitable for the deep learning mathematical model, in one application, it is possible to use one-hot encoding to represent the input sequences. Then, in one embodiment, the sequence encodings go through six convolutional layers and four pooling layers, which are designed to detect important motifs and aggregate both useful local and global information across the whole input sequence. The outputs of the last pooling layer are flattened and then go through three fully-connected layers, which are designed to learn the functional mapping between the learned representation from the convolutional layers and the final labeling space. Since all the tasks of the HMD-ARG model 110 are classification problems, regarding the ARG/non-ARG and the β-lactam subtype prediction, in this embodiment a standard cross-entropy loss function was used. The multi-task learning loss function is discussed later.

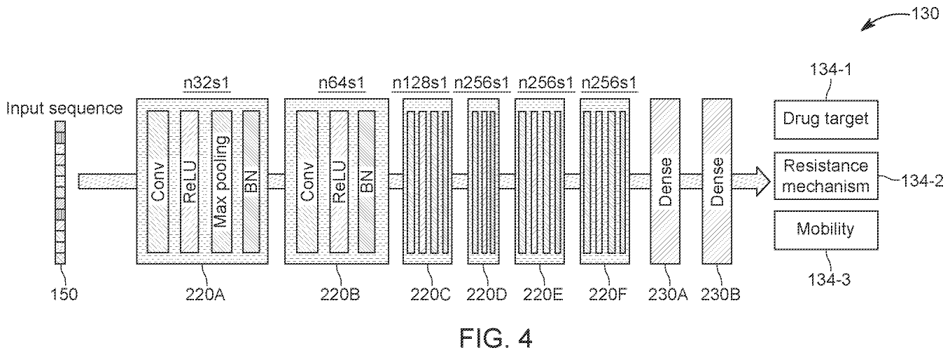

Within this framework, there is a level 1 model 130 performing multi-task learning for the coarse resistant drug type, functional mechanism and gene mobility prediction. The architecture for the level 1 model 130 is similar to that described with regard to FIGS. 2 and 3. However, as illustrated in FIG. 4, the level 1 model 130, instead of only having one fully-connected branch with Softmax activation function, has three fully-connected branches 134-1 to 134-3, which correspond to the three tasks, respectively. In other words, the level 1 model 130 for multi-task learning is essentially composed of three models, while those models share the convolutional layers 220A to 220F and the pooling layers. One advantage of this multi-task learning scenario is that the three tasks 134-1 to 134-3 can force those layers (convolutional and fully-connected layers discussed in FIG. 4) to discover distinct features within the input sequences, which are useful for all the three tasks, and thus prevent the model from overfitting. Note that the model shown in FIG. 4 has six convolution layers 220A to 220F and two fully-connected layers 230A and 230B while the model shown in FIG. 2 has five convolution layers and three fully-connected layers. The layers of the first and second convolution layers 220A and 220B are explicitly identified in FIG. 4 and also identified with different shades while the remaining four convolution layers have their layers identify only by the corresponding shades.

For this model, the loss function is modified as follows:

Lₘᵤₗₜᵢ₋ₜₐₛₖ=αL_{drug}+βL_{mechanism}+γL_{source} (1)

where α, β, and γ are the weights of each task and they are hyperparameters, and L_{drug}, L_{mechanism}, and L_{source} are the cross-entropy losses of the corresponding task. According to this embodiment, the model optimizes over the weighted Lₘᵤₗₜᵢ₋ₜₐₛₖ loss function instead of each cross-entropy loss alone, to take care of all the three tasks simultaneously. After training the above model, given an input sequence, it is possible to obtain prediction results of the three tasks with one single forward-propagation.

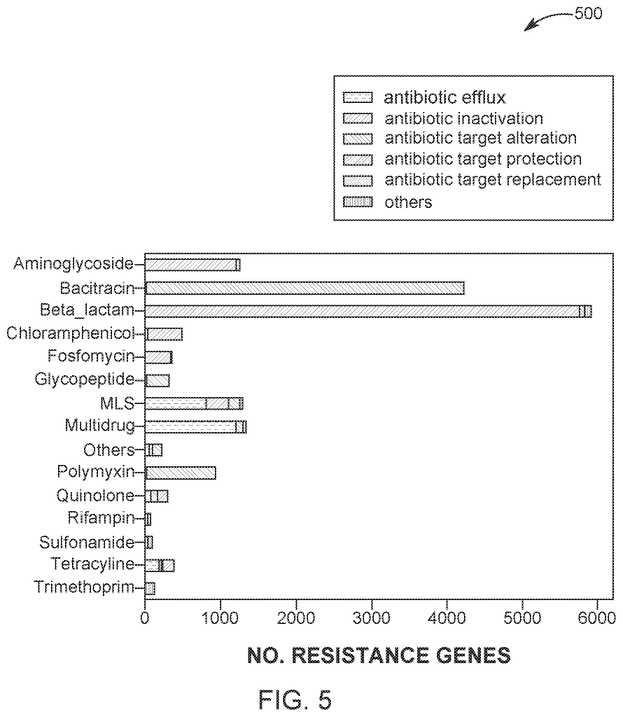

The HMD-ARG model 110 was tested with a database as now discussed. The inventors collected and cleaned antibiotic resistance genes from seven published ARG database. They are Comprehensive Antibiotic Resistance Database (CARD), AMRFinder, ResFinder, Antibiotic Resistance Gene-ANNOTation (Arg-ANNOT), DeepARG, MEGARes, and Antibiotic Resistance Genes Database (ARDB). The ARGs were assigned with three kinds of annotations: drug target, mechanism of antibiotic resistance, and transferable ability. For the drug target, the inventors adopted labels from their source databases. Experts in this filed decided the final label for conflict records. As for resistance mechanism annotation, the inventors used the ontology system from CARD, and assigned a mechanism label to the ARGs using the BLASTP and best-hit strategy with a cut-off score 1e−20. There are 1,994 sequences in this database that missed tags under this condition, so experts checked the original publications and assigned labels accordingly. The composition of this database 500 is shown in FIG. 5. The figure shows the total counts of ARGs categorized by their drug targets and resistance mechanisms. The number of ARGs is indicated on the x-axis, and inside each target drug, the resistance mechanisms are color-encoded in the key.

For the gene mobility type, the inventors used AMRFinder, the up-to-date acquired ARGs database for label annotation. The inventors used the command line tool offered by the AMRFinder, which includes both sequence alignment method and HMM profiles for discriminating gene transferable ability. Mobile genetic elements surrounding the ARGs are needed to be surveyed for further validation of predicted mobility of ARGs.

The level 1 module 130 in FIG. 1 is configured to indicate (1) the drug target, (2) the resistance mechanism, and (3) the transferable ability. With regard to the drug target, the discovery and synthesis of antibiotics in the past years has produced a large number of drugs, for instance, Penicillin, Cefazolin, etc. Using the list of all drugs as classes can be a choice, but it will introduce some unnecessary problems such as unbalanced dataset. Naturally, they can be grouped through their mechanisms of action. Aminoglycoside can inhibit protein synthesis while trimethoprim modifies the energy metabolism of microbial. According to this embodiment, various anatomical therapeutic chemical (ATC) classes from WHO (see Anatomical therapeutic chemical classification system, https://www. whocc.no/atc_ddd_index/?code=J01) are used. In this specific implementation, fifteen classes (listed on they axis in FIG. 5) are used for the drug target system. However, one skilled in the art would understand that more or less classes may be used. These 15 classes include macrolide-lincosamide-streptogramin (MLS), tetracycline, quinolone, aminoglycoside, bacitracin, beta-lactam, fosfomycin, glycopeptide, chloramphenicol, rifampin, sulfonamide, trimethoprim, polymyxin, multidrug, and others.

With regard to the resistance mechanism that is also determined by the level 1 module 130, it is noted that the bacteria have become resistant to antibiotics through several mechanisms. The Antibiotic Resistance Ontology (ARO) developed by CARD has a clear classification scheme on resistance mechanism annotation. They include seven classes: antibiotic target alteration, antibiotic target replacement, antibiotic target protection, antibiotic inactivation, antibiotic efflux, and others. In this embodiment, the inventors adopted the mechanism part of the ARO system and further combined the “reduced permeability to antibiotic” and the “resistance by absence” into “others” since they are both related to porin and appears less frequently in the database 500 illustrated in FIG. 5.

With regard to the transferable ability, the antibiotic resistance is ancient; wild type resistance gene exists for at least 30,000 years. It has been becoming a major concern since microorganisms can interchange resistance genes through horizontal gene transfer (HGT). Both ways can achieve resistance phenotypes, so it is desired to distinguish whether a resistance gene could transfer between bacteria. Roughly speaking, if a resistance gene is on a mobilizable plasmid, then it has the potential to transfer.

Beta-lactamases are bacterial hydrolases that bind an acylate beta-lactam antibiotics. There are mainly two mechanisms: the active-site serine beta-lactamases, and the Metallo-beta-lactamases that requires metal ion (e.g., Zn2+) for activity. Serine beta-lactamases could be further divided into class A, C, and D according to sequence homology. That is the same for Metallo-beta-lactamases, which can be divided into class B1, B2, and B3. This annotation is not explicitly shown in the database 500 shown in FIG. 5. The level 2 classification model 140 was trained based on the data found in Thierry et al. (2017) (Thierry Naas, Saoussen Oueslati, R'emy A Bonnin, Maria Laura Dabos, Agustin Zavala, Laurent Dortet, Pascal Retailleau, and Bogdan I lorga. Beta-lactamase database (bldb)—structure and function. Journal of enzyme inhibition and medicinal chemistry, 32(1):917-919, 2017). The classes for this data are as follow:

Class A: the active-site serine beta-lactamases, known primarily as penicillinases.

Representatives: TEM-1, SHV-1.

Class B: the Metallo-beta-lactamases (MBLs), have an extremely broad-spectrum substrate.

Class B1 representatives: NDM-1, VIM-2, IMP-1.

Class B2 representatives: CphA.

Class B3 representatives: L1.

Class C: the active-site serine beta-lactamases, tend to prefer cephalosporins as substrates.

Representatives: P99, FOX-4.

Class D: the active-site serine beta-lactamases, it's a diverse class, confer resistance to penicillins, cephalosporins, extended-spectrum cephalosporins, and carbapenems.

Representatives: OXA-1, OXA-11, CepA, KPC-2.

The inventors collected 66k non-ARGs from UniProt database that share high sequence similarity score with the ARGs from database 500, and then trained the level 0 model 120 on the combined dataset. The level 1 multi-task learning was implemented with the database 500.

For the β-lactamase subclass label, the HMD-ARG model 110 was trained on an up-to-date beta-lactamase database, BLDB. At each level, a CNN was used for the classification task.

First, each amino acid is converted into a one-hot encoding vector, then protein sequences are converted into a zero-padded numerical matrix of size 1576×23, where 1576 meets the length of longest ARGs and non-ARGs in the dataset 500, and 23 stands for 20 standard amino acids and two infrequent amino acids, B and Z. One more symbol X stands for unknown ones.

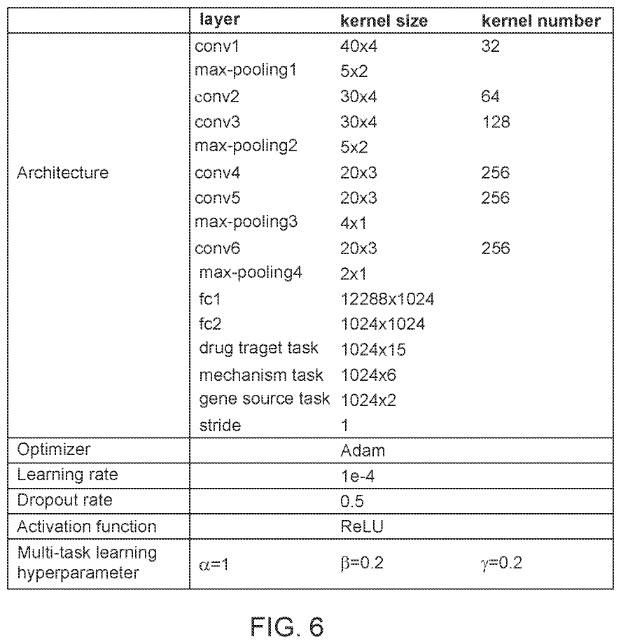

Such encoded matrix is then fed into the sequence of 6 convolutional layers and 4 max-pooling layers illustrated in FIG. 4. The parameters in the HMD-ARG model 110 involve model architecture, the kernel size and the number of kernels in the convolutional layers, the pooling kernel size of the max-pooling layer, dropout rate, the optimizer algorithm, and learning rate. A set of values for these parameters is illustrated in FIG. 6. Note that other values may be used for the hyperparameters of the CNN model shown in FIG. 4.

Because the focus in the level 1 model 130 is on the classification of all three tasks, the cross-entropy given by equation (1) is used as the loss function. Specifically, the level 1 model 130 performs multi-task learning for the drug target, mechanism of antibiotic resistance, and transferable ability simultaneously with a weighted sum loss function on the three tasks as discussed above.

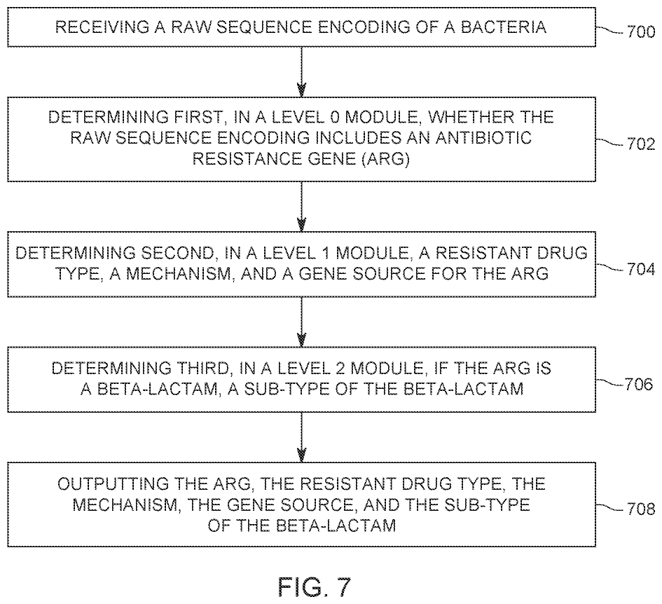

A method for annotating antibiotic resistance genes based on the HMD-ARG model introduced above is now discussed with regard to FIG. 7. The method includes a step 700 of receiving a raw sequence encoding 102 of a bacteria, a step 702 of determining first, in a level 0 module 120, whether the raw sequence encoding 102 includes the ARG, a step 704 of determining second, in a level 1 module 130, a resistant drug type, a mechanism, and a gene mobility for the ARG, a step 706 of determining third, in a level 2 module 140, if the ARG is a beta-lactam, a sub-type of the beta-lactam, and a step 708 of outputting the ARG, the resistant drug type, the mechanism, the gene mobility, and the sub-type of the beta-lactam. The level 0 module 120, the level 1 module 130 and the level 2 module 130 each includes a deep CNN model 200.

In one application, the CNN model includes a single output for the level 0 module and the level 2 module and three outputs for the level 1 module. In this or another application, the CNN model includes six convolutional layers, four max-pooling layers, and two fully-connected layers for each of the level 0 module, level 1 module and level 2 module. The CNN model applies a one-hot encoding to the received raw sequence encoding.

The method may further include a step of applying a cross-entropy as a loss function for simultaneously determining the resistant drug type, the mechanism, and the gene mobility. In one application, the CNN model operates directly on the raw sequence encoding. The steps of determining first, determining second, and determining third do not utilize sequence alignment.

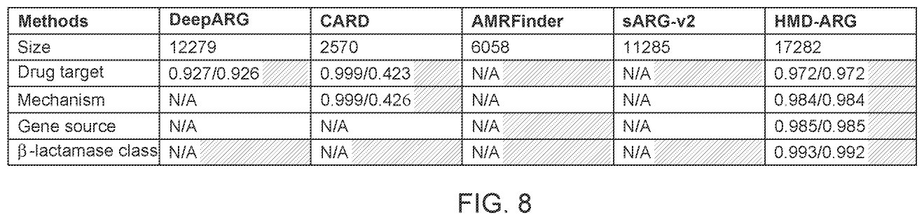

The performance of the HMD-ARG model 110 is now discussed with regard to the table in FIG. 8. The table summarizes the performance comparison between four different existing tools and the HMD-ARG model 110, for different tasks.

The first two rows indicate the name of the database and the database size (up to July 2019) while the rest of the four rows gray code the cells to indicate whether the database includes that annotation or not, and the number inside each cell is the precision/recall score in cross-validation experiments. The symbol “N/A” means that the tool is unable to perform that task directly. For example, the tool sARG-v2 is designed for raw reads rather than assembly sequence that were studied herein and thus, this tool cannot perform any determination of the assembly sequence. The database 500 has the largest size and the model performs well, achieving a high rate in all the tested tasks.

The comparison between the HMD-ARG model 110 and the other four models noted in FIG. 8 is now discussed in more detail. The DeepARG is a deep learning model for antibiotic resistance annotation. It takes protein sequences as inputs, then compares the sequence with the self-curated database and uses a dissimilarity score as deep learning model inputs. Model outputs are drug targets. The DeepARG can be found at https://bench.cs.vt.edu/deeparg. In terms of similarities, this model uses deep learning models for gene annotation. Both this method and the model HMD-ARG 100 assign gene annotation on the assembly sequences level rather than the raw reads from sequencing data, and the outputs contain resistance drug target. Part of the HMD-ARG database sequence comes from DeepARG. However, in terms of differences, the DeepARG uses a sequence dissimilarity score as deep learning model inputs. The HMD-ARG model is an end-to-end model, uses one-hot encoding sequence directly, and the outputs contain more annotation than other drug targets. The deep learning model structure is also different. The DeepARG is a multi-layer perception, while the HMD-ARG model includes a CNN-based model. In terms of advantages, the level 0 of the DeepARG model is a sequence alignment method based on a cut-off score, while the HMD-ARG model 110 is a deep learning model. The HMD-ARG model provides hierarchical outputs, while the DeepARG can only predict the drug target. The performance of the HMD-ARG model is better than the DeepARG model.

In terms of the CARD model, CARD is an ontology-based database that provides comprehensive information on antibiotic resistance genes and their resistance mechanisms. It also applies a sequence alignment-based tool (RGI) for target prediction. This database can be found at https://card.mcmaster.ca/. The resistance mechanism label of the HMD-ARG model adopts the CARD's ontology system, and all CARD database sequences are in the HMD-ARG database. Both methods take assembly sequence as inputs. However, these two models are different as the RGI tool is a pairwise comparison method based on the sequence alignment method, and the result will be influenced largely by a cut-off score, while the HMD-ARG model is an end-to-end deep learning model. Thus, the RGI tool predicts level 0 and level 1 simultaneously with the sequence alignment method, which requires a manually cut-off score, which is prone to many false-negative results, a situation that is avoided by the configuration of the HMD-ARG model.

In terms of the AMRFinder, the AMRFinder can identify acquired antibiotic resistance genes in either protein datasets or nucleotide datasets, including genomic data. The AMRFinder relies on NCBI's curated AMR gene database and the curated collection of Hidden Markov Models. The AMRFinder can be found at https://www.ncbi.nlm.nih.gov/pathogens/antimicrobial-resistance/AMRFinder/. The intrinsic/acquired label in the HMD-ARG model is labeled by the AMRFinder. All sequences in the AMRFinder are present in the HMD-ARG model. However, the AMRFinder is a pairwise comparison method based on self-curated antimicrobial genes and Hidden Markov Models, so it has some manually cut-off on thresholds, while the HMD-ARG model is an end-to-end deep learning model and does not require any cut-off. The AMRFinder does not explicitly offer drug target and mechanism label. Thus, given an input sequence, the AMRFinder only provides the best-hit sequence in its database with sequence alignment method and HMM profiles. The HMD-ARG model could give the labels directly without using sequence alignment, which is advantageous.

In terms of the sARG-v2, sARG-v2, or ARGs-OAP v2.0, sARG-v2 is a database that contains sequences from CARD, ARDB and AMRFinder databases. It also provides self-curated Hidden Markov Model profiles of ARG subtypes. The sARG-v2 can be found at https://smile.hku.hk/SARGs. The sARG-v2 and HMD-ARG databases share similar ARG sequences, and both databases have a hierarchical structure on annotations. However, ARGs-OAP v2.0 works on metagenomic data, the input is raw-reads directly, while the HMD-ARG model is an assembly-based method. The ARGs-OAP v2.0 classify sequences according to curated HMM profiles, while the HMD-ARG model is an end-to-end deep learning model. Thus, the ARGs-OAP v2.0 does not work on assembly sequences, as the HMD-ARG model.

As suggested by the table in FIG. 8, with the help of the novel hierarchical multi-task learning design and the deep learning model used by the HMD-ARG model 110, the proposed method can achieve the state-of-the-art performance on all the three annotation tasks. Furthermore, the database 500 that was generated by the inventors along with the server is the most comprehensive one, with all the three pieces of labeling information. Further, the server 112 discussed with regard to FIG. 1, in which the HMD-ARG model 110 is implemented, allows the users to submit the protein sequence without any other further configurations and the result would be returned back in around one minute. Thus, the HMD-ARG model 110 is not only better than the other existing tools, but also fast.

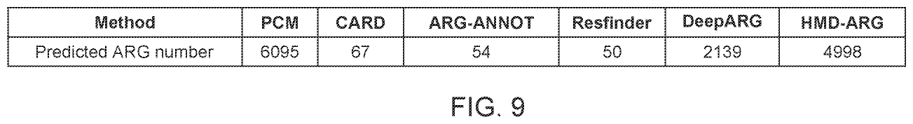

The performance of the HMD-ARG model was further tested by analyzing data from two independent studies. A first validation data comes from a three-dimensional, structure-based method (PCM) prediction result in a catalog of 3.9 million proteins from human intestinal microbiota. Though not all the predictions are experimentally validated, it utilizes structure information and is supposed to be more accurate. The inventors collected the 6,095 antibiotic resistance determinants (ARDs) sequences predicted by the PCM method and compared the results of the ARG/non-ARG prediction performance of the HMD-ARG model with other models, as illustrated in FIG. 9. The results presented in FIG. 9 indicate that the HMD-ARG model by far outperforms the existing tools.

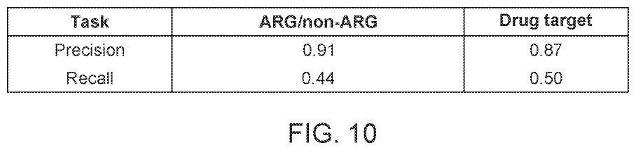

A second validation data comes from different North American soil samples and have been experimentally validated with functional metagenomics approach. The inventors have collected protein sequence from GenBank(KJ691878-KJ696532), removed duplicated genes that also appeared in the database 500, and chose the relevant ARGs according to the antibiotics used for the screening of the clones: beta-lactam, aminoglycoside, tetracycline, trimethoprim. According to the paper and gene annotations, the inventors obtained 2,050 ARGs with these four drug target label and 1,992 non-ARGs. The performance of the level 0 and level 1 modules of the HMD-ARG model 110 are illustrated in FIG. 10 and indicate that although the model 110 still faced many false negatives, its precision rate is high. The results suggest the ability of the HMD-ARG model 110 to annotate resistance genes.

As discussed above, the abuse of antibiotics in the last several decades has given rise to antibiotic resistance, that is, an increasing number of drugs are losing sensitivity to bacteria that they were designed to kill. An essential step for fighting against this crisis is to track the potential source and exposure pathway of antibiotic resistance genes in clinical or environmental samples. While traditional methods like antimicrobial susceptibility testing (AST) can provide insights into the prevalence of the antimicrobial resistance, they are both time- and resource-consuming, and thus cannot handle the diverse and complex microbial community. Existing tools based on sequence alignment or motif detection often have a high false negative rate and can be biased to specific types of ARGs due to the incompleteness of ARGs databases. As a result, they are often unsuccessful in characterizing the diverse group of ARGs from metagenomic samples. In addition, as discussed above, most existing computational tools do not provide information about the mobility of genes and the underlying mechanism of the resistance. To address those limitations, the HDM-ARG model 110 discussed above is an end-to-end hierarchical multi-task deep learning framework for antibiotic resistance gene annotation, taking raw sequence encoding as input and then annotating ARGs sequences from three aspects: resistant drug type, the underlying mechanism of resistance, and gene mobility. To the best of the inventors' knowledge, this tool is the first one that combines ARG function prediction with deep learning and hierarchical classification

Antibiotic resistance genes annotation tools are crucial for clinical settings. The server discussed with regard to FIG. 1 can take the new gene sequences as inputs, extract data-specific features, and perform functional prediction for those new genes from multiple perspectives. There are two potential ways of using the server illustrated in FIG. 1. Firstly, for a given gene, the tool can determine whether the gene is an ARG or not, and if the gene is an ARG, what drugs can the gene resist at. By providing such information to the clinician, it can help the clinician to suggest more effective drugs to the patients accordingly, avoiding the usage of those drugs which have lost its effectiveness because of antibiotic resistance. Secondly, this tool could provide an initial function checking of the newfound or widely spread ARGs, which are critical for the hospital and farms. By reducing the time and money spent on the finding of the potential source of resistance genes, it can facilitate the analysis of the detailed composition of resistant metagenomic samples, as well as the study of the possible way for preventing the resistance gene spreads

Thus, given an input protein sequence, the HMD-ARG model 110 first predicts the ARG or non-ARG using the level 0 module 120. If it is ARG, the level 1 module 130 predicts the three annotations mentioned above, i.e., drug target, resistance mechanism, and transferable ability. Specifically, if it could resist β-lactam, the level 2 module 140 further predicts its subclass label.

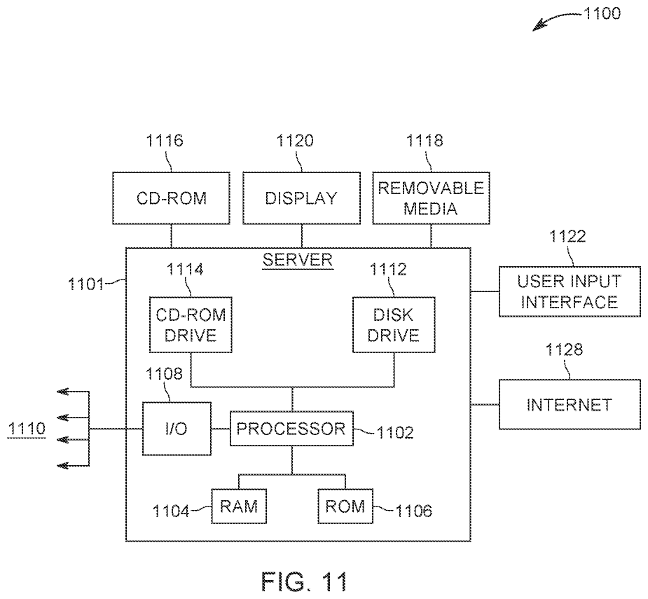

The above-discussed modules and methods may be implemented in a server as illustrated in FIG. 11. Hardware, firmware, software or a combination thereof may be used to perform the various steps and operations described herein. A computing system 1100 suitable for performing the activities described in the exemplary embodiments may include a server 112. Such a server 112 may include a central processor (CPU) 1102 coupled to a random-access memory (RAM) 1104 and to a read-only memory (ROM) 1106. ROM 1106 may also be other types of storage media to store programs, such as programmable ROM (PROM), erasable PROM (EPROM), etc. Processor 1102 may communicate with other internal and external components through input/output (I/O) circuitry 1108 and bussing 1110 to provide control signals and the like. Processor 1102 carries out a variety of functions as are known in the art, as dictated by software and/or firmware instructions.

Server 1101 may also include one or more data storage devices, including hard drives 1112, CD-ROM drives 1114 and other hardware capable of reading and/or storing information, such as DVD, etc. In one embodiment, software for carrying out the above-discussed steps may be stored and distributed on a CD-ROM or DVD 1116, a USB storage device 1118 or other form of media capable of portably storing information. These storage media may be inserted into, and read by, devices such as CD-ROM drive 1114, disk drive 1112, etc. Server 1101 may be coupled to a display 1120, which may be any type of known display or presentation screen, such as LCD, plasma display, cathode ray tube (CRT), etc. A user input interface 1122 is provided, including one or more user interface mechanisms such as a mouse, keyboard, microphone, touchpad, touch screen, voice-recognition system, etc.

Server 1101 may be coupled to other devices, such as sources, detectors, etc. The server may be part of a larger network configuration as in a global area network (GAN) such as the Internet 1128, which allows ultimate connection to various landline and/or mobile computing devices.

The disclosed embodiments provide a model and a server that can determine whether the gene is an ARG or not, and if the gene is an ARG, what drugs can the gene resist. It should be understood that this description is not intended to limit the invention. On the contrary, the embodiments are intended to cover alternatives, modifications and equivalents, which are included in the spirit and scope of the invention as defined by the appended claims. Further, in the detailed description of the embodiments, numerous specific details are set forth in order to provide a comprehensive understanding of the claimed invention. However, one skilled in the art would understand that various embodiments may be practiced without such specific details.

Although the features and elements of the present embodiments are described in the embodiments in particular combinations, each feature or element can be used alone without the other features and elements of the embodiments or in various combinations with or without other features and elements disclosed herein.

This written description uses examples of the subject matter disclosed to enable any person skilled in the art to practice the same, including making and using any devices or systems and performing any incorporated methods. The patentable scope of the subject matter is defined by the claims, and may include other examples that occur to those skilled in the art. Such other examples are intended to be within the scope of the claims.

### REFERENCES

Arango-Argoty, G., Garner, E., Pruden, A., Heath, L. S., Vikesland, P., and Zhang, L. (2018). Deeparg: a deep learning approach for predicting antibiotic resistance genes from metagenomic data. Microbiome, 6(1), 23.
Feldgarden, M., Brover, V., Haft, D. H., Prasad, A. B., Slotta, D. J., Tolstoy, I., Tyson, G. H., Zhao, S., Hsu, C.-H., McDermott, P. F., et al. (2019). Using the ncbi amrfinder tool to determine antimicrobial resistance genotype-phenotype correlations within a collection of narms isolates. BioRxiv, page 550707.
Yin, X., Jiang, X.-T., Chai, B., Li, L., Yang, Y., Cole, J. R., Tiedje, J. M., and Zhang, T. (2018). Args-oap v2. 0 with an expanded sarg database and hidden markov models for enhancement characterization and quantification of antibiotic resistance genes in environmental metagenomes. Bioinformatics, 34(13), 2263-2270.
Gupta, S. K., Padmanabhan, B. R., Diene, S. M., Lopez-Rojas, R., Kempf, M., Landraud, L., and Rolain, J.-M. (2014). Arg-annot, a new bioinformatic tool to discover antibiotic resistance genes in bacterial genomes. Antimicrobial agents and chemotherapy, 58(1), 212-220.
Li, Y., Huang, C., Ding, L., Li, Z., Pan, Y., and Gao, X. (2019). Deep learning in bioinformatics: Introduction, application, and perspective in the big data era. Methods.
Zou, Z., Tian, S., Gao, X., and Li, Y. (2019). mldeepre: Multi-functional enzyme function prediction with hierarchical multi-label deep learning. Frontiers in Genetics, 9, 714.
Krizhevsky A., Sutskever I., and Hinton G. Imagenet classification with deep convolutional neural networks. In Advances in neural information processing systems, pages 1097-1105, 2012.

1. A method for annotating antibiotic resistance genes, the method comprising:
receiving a raw sequence encoding of a bacterium;
determining first, in a level 0 module, whether the raw sequence encoding includes an antibiotic resistance gene (ARG);
determining second, in a level 1 module, a resistant drug type, a resistance mechanism, and a gene mobility for the ARG;
determining third, in a level 2 module, if the ARG is a beta-lactam, a sub-type of the beta-lactam; and
outputting the ARG, the resistant drug type, the resistance mechanism, the gene mobility, and the sub-type of the beta-lactam,
wherein the level 0 module, the level 1 module and the level 2 module each includes a deep convolutional neural network (CNN) model.

2. The method of claim 1, wherein the CNN model includes a single output for the level 0 module and the level 2 module and three outputs for the level 1 module.

3. The method of claim 1, wherein the CNN model includes six convolutional layers, four max-pooling layers, and two fully-connected layers for each of the level 0 module, level 1 module and level 2 module.

4. The method of claim 1, wherein the CNN model applies a one-hot encoding to the received raw sequence encoding.

5. The method of claim 1, further comprising:
applying a cross-entropy as a loss function for simultaneously determining the resistant drug type, the resistance mechanism, and the gene mobility.

6. The method of claim 1, wherein the CNN model operates directly on the raw sequence encoding.

7. The method of claim 1, wherein the steps of determining first, determining second, and determining third do not utilize sequence alignment.

8. A server for annotating antibiotic resistance genes, the server comprising:
an interface for receiving a raw sequence encoding of a bacterium; and
a processor connected to the interface and configured to,
determine first, in a level 0 module, whether the raw sequence encoding includes an antibiotic resistance gene (ARG);
determine second, in a level 1 module, a resistant drug type, a mechanism, and a gene mobility for the ARG;
determine third, in a level 2 module, if the ARG is a beta-lactam, a sub-type of the beta-lactam; and
output the ARG, the resistant drug type, the mechanism, the gene mobility, and the sub-type of the beta-lactam,
wherein the level 0 module, the level 1 module and the level 2 module each includes a deep convolutional neural network (CNN) model.

9. The server of claim 8, wherein the CNN model includes a single output for the level 0 module and the level 2 module and three outputs for the level 1 module.

10. The server of claim 8, wherein the CNN model includes six convolutional layers, four max-pooling layers, and two fully-connected layers for each of the level 0 module, level 1 module and level 2 module.

11. The server of claim 8, wherein the CNN model applies a one-hot encoding to the received raw sequence encoding.

12. The server of claim 8, wherein the processor is further configured apply a cross-entropy as a loss function for simultaneously determining the resistant drug type, the mechanism, and the gene mobility.

13. The server of claim 8, wherein the CNN model operates directly on the raw sequence encoding.

14. The server of claim 8, wherein the steps of determining first, determining second, and determining third do not utilize sequence alignment.

15. A hierarchical, multi-task, deep learning model for annotating antibiotic resistance genes, the model comprising:
an input for receiving a raw sequence encoding of a bacterium;
a level 0 module configured to determine first, whether the raw sequence encoding includes an antibiotic resistance gene (ARG);
a level 1 module configured to determine second, a resistant drug type, a mechanism, and a gene mobility for the ARG;
a level 2 module configured to determine third, if the ARG is a beta-lactam, a sub-type of the beta-lactam; and
an output configured to output the ARG, the resistant drug type, the mechanism, the gene mobility, and the sub-type of the beta-lactam,
wherein the level 0 module, the level 1 module and the level 2 module each includes a deep convolutional neural network (CNN) model.

16. The model of claim 15, wherein the CNN model includes a single output for the level 0 module and the level 2 module and three outputs for the level 1 module.

17. The model of claim 15, wherein the CNN model includes six convolutional layers, four max-pooling layers, and two fully-connected layers for each of the level 0 module, level 1 module and level 2 module.

18. The model of claim 15, wherein the CNN model applies a one-hot encoding to the received raw sequence encoding.

19. The model of claim 15, further comprising:
applying a cross-entropy as a loss function for simultaneously determining the resistant drug type, the mechanism, and the gene mobility.

20. The model of claim 15, wherein the CNN model operates directly on the raw sequence encoding, and wherein the steps of determining first, determining second, and determining third do not utilize sequence alignment.
